(19) United States
(12) Patent Application Publication
(10) Pub. No.: US 2002/0183728 A1
(43) Pub. Date: Dec. 5, 2002

(54) Laser probe

(76) Inventors: Yitzhak Rosenberg, Ramat Gan (IL); Ra?apos;anan Yemini, Kfar Vitkin (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 10/142,934

(22) Filed: May 13, 2002

Related U.S. Application Data
(60) Provisional application No. 60/290,348, filed on May 14, 2001.

(51) Int. Cl. A61B 18/20

(52) U.S. Cl. 606/15; 606/16; 606/17

(57) ABSTRACT

A laser probe has a central channel running substantially the length of the probe from one portion to a distal portion with an angled-cut section. The angled-cut section has a diameter sufficiently small to be inserted between a tooth surface and adjacent gum tissue. The channel may either include an optical fiber or may be hollow.

### FIELD OF THE INVENTION

The present invention relates to the field of laser probes in general. More specifically, the present invention relates to laser probes for dental applications.

### BACKGROUND OF THE INVENTION

There are several known methods for removal of calculus from teeth. One common method uses mechanical instruments such as a scrapper to mechanically remove the calculus from the patient's teeth. This method may be painful, and usually does not allow for completely effective removal of the calculus as the mechanical tools are not capable of accessing all the areas on a tooth where calculus may reside.

Another common method for calculus removal uses an ultrasonic (Ultrasound) therapeutic apparatus. As part of the ultrasound therapy, a probe produces a focused ultrasonic beam whose shock waves break the calculus. Since probes available for use with the ultrasonic therapeutic apparatus can not reach all the areas on the tooth where calculus may reside (e.g the area between the tooth and the gum, as shown in FIG. 1.), the ultrasonic method suffers from many of the same disadvantages as the mechanical method.

Generally, the probes available today for use in dental procedures, such as calculus removal, do not allow for easy access to the cervical zone between the tooth and the gum.

### SUMMARY OF THE INVENTION

The present invention provides a laser probe, having an angled-cut front portion, thus allowing the probe access to the cervical zone. The probe may be used for many dental applications, such as removal of calculus, decay or other residuals from any surface of a tooth, including the root surfacing and the tubules sealing.

According to one embodiment of the present invention, the range of the angle of the cut in the front portion of the probe may be between 20-45°.

According to a further embodiment of the present invention, the probe may be a hollow wave-guide.

According to additional embodiments of the present invention, the probe may be used in conjunction with a CO2 laser, an Er:YAG laser, an Er:YSGG laser or any other desirable laser.

### BRIEF DESCRIPTION OF THE DRAWINGS

The present invention may be understood and appreciated more fully from the following detailed description taken in conjunction with the appended drawings in which:

FIG. 1 is a schematic illustration of the area between the teeth and the gum;

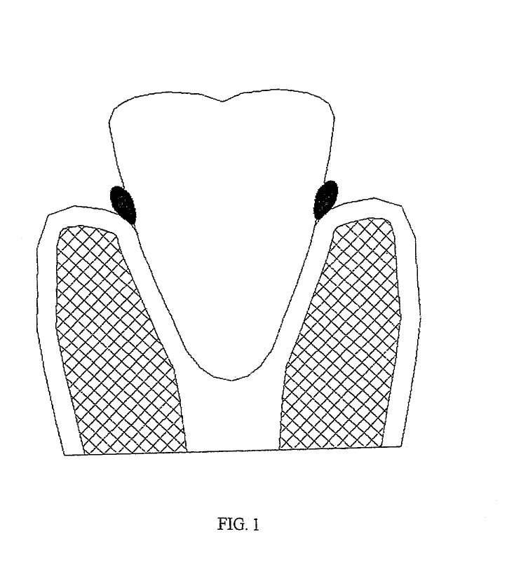

FIG. 2 is a side cross sectional illustration of a probe according to some embodiments of the present invention;

FIG. 3 is a side view of a probe of FIG. 2;

FIG. 4 is an illustration of the use of the probe according to some embodiments of the present invention;

FIG. 5 is a schematic illustration of the passage of the radiation rays inside a probe according to some embodiments of the present invention;

FIG. 6 is a schematic illustration of an additional probe according to some embodiments of the present invention; and

FIG. 7 is a schematic illustration of a further additional probe according to some embodiments of the present invention.

It will be appreciated that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to indicate corresponding or analogous elements.

### DETAILED DESCRIPTION OF THE PRESENT INVENTION

The present invention provides a laser probe having an angled-cut front portion. As part of the present invention, the probe may be used to access the cervical zone. The probe may be used for many dental applications, such as removal of calculus, decay or other residuals from tooth surfaces, including the root surface and the tubules sealing.

According to some embodiment of the present invention, the range of the angle of the cut in the front portion of the probe may be between 20-45°.

According to a further embodiment of the present invention, the probe may be a hollow wave-guide.

According to additional embodiments of the present invention, the probe may be used in conjunction with a CO2 laser, an Er:YAG laser, an Er:YSGG laser or any other desirable laser.

According to yet a further embodiment of the present invention, the probe may focus and project a treatment laser beam substantially parallel to a desired treatment surface.

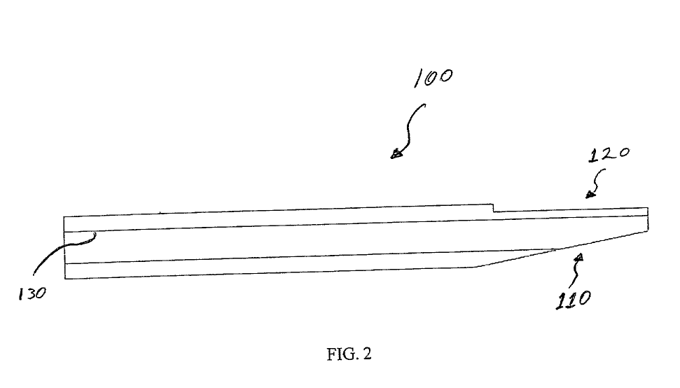

Reference is now made to FIG. 2, which is a side cross sectional view of a probe according to some embodiments of the present invention. The probe 100 may have a distal portion with an angled cut 110 and a depressed portion 120. The probe 100 may have an inner channel with an inner wall 130. The channel may either be hollow or may include an optical fiber. The channel may have a central axis substantially parallel to the inner wall. In one embodiment of the present invention, the inner wall 130 may be optically reflective.

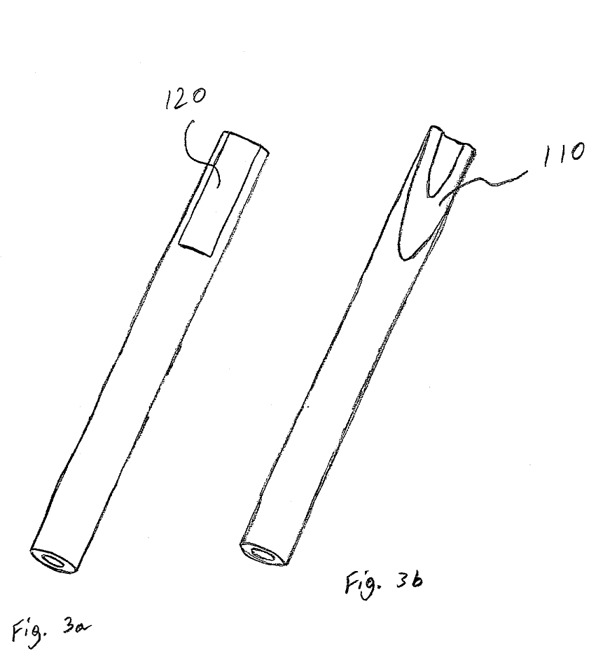

Turning now to FIGS. 3a and 3b, there are shown a top and a bottom isometric view of a probe according to some embodiments of the present invention. FIG. 3a shows an isometric view of a depressed section 120 of a probe according to the present invention, while FIG. 3b shows an isometric view of the angled-cut 110 section of a probe according to the present invention. As shown in these FIGS. 3a and 3b, the front or distal portion of the probe may be cut in one direction at an angle suitable to allow for efficient access to the cervical zone. The probe may be designed such that it can be placed easily between the tooth and the gum without creating a substantial gap between the two and without causing bleeding of the area or affecting the healthy tissue. The cut in the distal or front portion of the probe may be in the range of 20-45° to the probe.

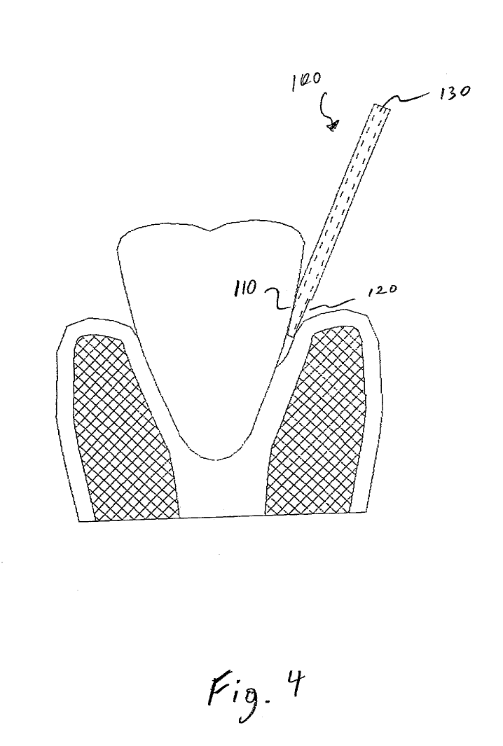

Turning now to FIG. 4, there is shown how a probe according to some embodiments of the present invention may be placed between a tooth and its adjacent gum. The section of the probe with the angled-cut 110 may be positioned adjacent to the tooth, while the depressed section of the probe may be adjacent to the gum.

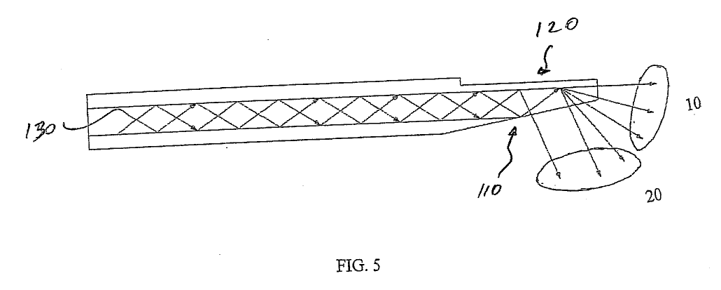

Turning now to FIG. 5, there is shown light guided inside a channel of a probe according to some embodiments of the present invention, and light radiating out the distal portion of the probe. The radiation source may be any laser. For applications relating to calculus removal and other hard tissue applications, lasers such as Er:YAG, CO2 or Er:YSGG may be used. However, the present invention is not limited to such lasers, rather some embodiments of the present invention may utilize numerous other lasers suitable for a wide variety of dental and non-dental applications.

The direction of the radiation emitted from the probe depends on the reflection angle of the radiation inside the probe. The reflection angle may be designed such that part of the radiation is used for crumbling calculus, and part of the radiation may be used for peeling calculus from the tooth. It would be obvious to those with ordinary skill in the art to alter one or more operation parameters of some embodiments of the present invention, such as the reflection angle, the wavelength of the laser radiation, the intensity of the laser radiation, the pulse duration, the spot size, etc., to configure suitable protocols for performing a multiplicity of treatments.

In this FIG. 5, the laser beams noted as entering region 10 are effective for peeling while the laser beams noted as entering region 20 are effective for crumbling. The angled-cut 110 in the front portion of the probe may direct the radiation emitted through the probe to a desired direction, e.g. the direction of the tooth where the calculus or other unwanted matter lies. The laser light emitted from the probe may radiate in a direction substantially parallel to a central axis of the probe's channel and/or the surface of the tooth to be treated. The majority of the radiation may be emitted to the direction of region 10. A smaller amount of radiation may be radiate in the direction of region 20. The vector sum of the total radiation emitted from the probe may be substantially parallel to the central axis of the probe's channel 130, with a small vector component in a direction perpendicular to the plane defined by the angled-cut 110. The required energies for the removal of calculus, for example, are relatively low. When an Er:YAG laser is used, for example, the required energy is in the range of 100 Joules.

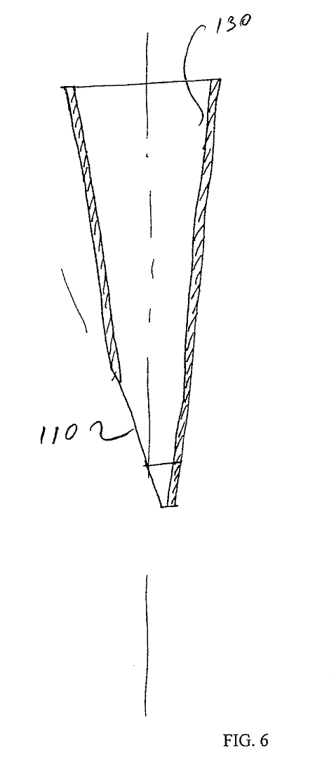

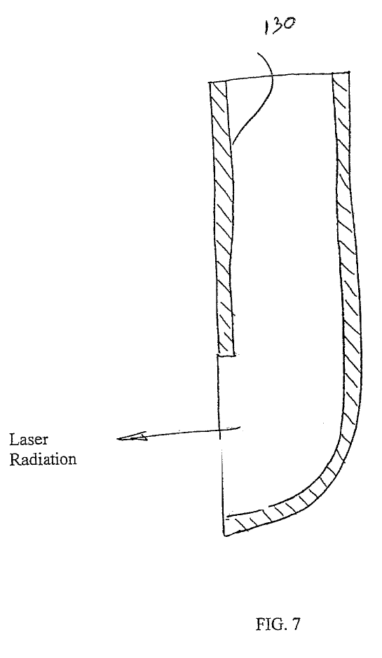

FIGS. 6 and 7 schematically illustrate additional embodiments of laser probes according to some embodiments of the present invention. FIG. 6 shows a side cross sectional view of a sharpened laser probe having an angled-cut 110 front portion. FIG. 7 shows a cross sectional view of a laser probe having its front portion curved, bent or otherwise configured such that the laser radiation is directed generally perpendicular to the probe. Such a probe may be used for directing laser radiation substantially perpendicular to a central axis of the probe's channel and the surface of a tooth. It should be appreciated by one of ordinary skill in the art, that numerous other probe configurations may be designed for performing a wide variety of tasks.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A laser probe comprising;
a channel running substantially the length of the probe;
an angled-cut section on the distal portion, said angled-cut section having a diameter sufficiently small to be inserted between a tooth surface and adjacent gum tissue.

2. The laser probe according to claim 1, wherein said angled-cut section has an angle to the probe in the range of 20 to 45 degrees.

3. The laser probe according to claim 1, further comprising a depressed section on the distal portion of said probe.

4. The laser probe according to claim 1, wherein said channel is comprised of fiber optical cable.

5. The laser probe according to claim 1, wherein said channel is a hollow cavity.

6. The laser probe according to claim 5, wherein said channel includes an optically reflective wall.

7. The laser probe of claim 1, wherein said laser probe is adapted to guide a laser beam produced by a group of lasers consisting of a CO₂ laser, an Er:YAG laser and an Er:YSGG laser.

8. The laser probe of claim 1 wherein the distal section of said laser probe is blocked, thereby not allowing electromagnetic radiation to be emitted in a direction substantially parallel to a central axis substantially parallel to the inner wall of said probe.

9. A laser probe comprising;
a channel running substantially the length of said probe, the distal portion of said probe being curved in a direction substantially perpendicular to a central axis of the channel and having an opening substantially parallel to the central axis of the channel.

10. The laser probe according to claim 9, wherein said curved distal portion has an angle to the channel axis ranging from 45 to 90 degrees.

11. The laser probe according to claim 9, wherein said channel is comprised of fiber optical cable.

12. The laser probe according to claim 9, wherein said channel is a hollow cavity.

13. The laser probe according to claim 12, wherein said channel includes an optically reflective wall.

14. The laser probe of claim 9 wherein said laser probe is adapted to guide a laser beam produced by a group of lasers consisting of a CO₂ laser, an Er:YAG laser and an Er:YSGG laser.
